(19) United States
(12) Patent Application Publication
(10) Pub. No.: US 2024/0285646 A1
(43) Pub. Date: Aug. 29, 2024

(54) METHODS AND COMPOSITIONS FOR LIPID FORMULATION OF LIPOPHILIC SMALL MOLECULE THERAPIES OF THE HETEROCYCLIC TYPE

(71) Applicant: Neuronascent, Inc., Clarksville, MD (US)

(72) Inventor: Judith KELLEHER-ANDERSSON, Mountain View, CA (US)

(21) Appl. No.: 18/568,686

(22) PCT Filed: Jun. 10, 2022
(86) PCT No.: PCT/US2022/032955

§ 371 Date: Dec. 8, 2023

Related U.S. Application Data
(60) Provisional application No. 63/340,230, filed on May 10, 2022.
(60) Provisional application No. 63/209,841, filed on Jun. 11, 2021.

(51) Int. Cl. A61K 31/551; A61K 9/10; A61K 31/496; A61K 47/14; A61K 47/44

(52) U.S. Cl. CPC A61K 31/551; A61K 9/10; A61K 31/496; A61K 47/14; A61K 47/44

(57) ABSTRACT

Methods and compositions for delivery of a therapeutic useful for e.g., neuron regeneration, neuroprotection, and/or slowing disease progression. Treatment of diseases and conditions characterized by neurodegeneration.

### CROSS REFERENCE TO RELATED APPLICATIONS

This application claims the benefit of U.S. Provisional Patent Application No. 63/209,841, filed Jun. 11, 2021, and U.S. Provisional Patent Application No. 63/340,230, filed May 10, 2022, the entire contents of which are hereby incorporated by reference herein.

### FIELD

Some embodiments of the present invention generally relate to the field of Neurology and Neuropsychiatric disorders. More specifically, certain embodiments of the present invention provide methods and compositions for formulation of heterocyclics. In some aspects, the disclosed formulations can prevent, slow progression and/or reverse neurodegenerative disease.

### BACKGROUND

Alzheimer's disease is a brain disorder that gradually destroys neurons. Over 4.5 million people in America suffer from Alzheimer's, which mostly occurs in older adults. The risk of developing Alzheimer's disease approximately doubles every five years after age 65 and reaches to 50 percent by age 85. Patients afflicted with Alzheimer's disease lose their ability to learn, remember, reason, make decisions, communicate and carry out daily activities. The direct and indirect cost of caring for Alzheimer's disease patients has increased to at least $100 billion annually.

Stroke, and Parkinson's disease can also cause neuronal loss and lead to behavioral decline. Hearing loss caused by injury, aging and pharmaceutical use stems from cochlear neuron loss.

A number of developmental delay disorders, including Down syndrome and Fragile X syndrome, may be caused by insufficient hippocampal neurogenesis. Therapies that promote hippocampal neurogenesis may be effective in improving behavioral phenotypes of Down syndrome and Fragile X syndrome.

There is a need for pharmaceutical formulations providing improved delivery of active pharmaceutical ingredients (API) to target areas. More particularly, there is a need for improved methods and compositions comprising formulation for delivering an API through the blood brain barrier. There is also a need for compositions capable of improving long-term absorption to promote efficacy and to allow increased dosages and stability

### SUMMARY

In accordance with some aspects, the present invention provides methods and compositions comprising formulations useful for delivering an API across the blood brain barrier. In some aspects, the disclosed compositions provide improved long-term absorption to promote efficacy. In some aspects, the compositions may allow for increased dosages and stability.

The methods and compositions disclosed herein are particularly useful in the treatment of neurodegenerative diseases like Alzheimer's disease and neuropsychiatric conditions such as depression. The methods and compositions could also be suitable for the manufacture of research products either as one composition or as a mixture of compositions. The methods and compositions comprising compounds are also useful for inhibiting neuronal degeneration. Thus, in some aspects, the present invention finds particular utility in the treatment of diseases and conditions characterized by neuronal loss including, but not limited to, Alzheimer's disease, Parkinson's disease, traumatic brain injury, and hearing loss. Disclosed herein are representative formulations, methods for making the compound product, compositions comprising the compounds, and methods for using the compounds.

In one aspect, the present invention provides compositions comprising compounds useful for treating humans, particularly aged humans, with or without neurodegenerative disorders.

In another aspect, the present invention is further directed to methods and compositions comprising compounds that have utility in the treatment of any diseases associated with neuron loss. More specifically, the present invention further provides methods for administering API in lipid formulation administered to a subject, such as an aged human. In one embodiment, the method may comprise administering to an aged human a liquid formulation. The composition comprising a compound described herein may be administered in an amount effective to be safely absorbed and stimulate neuron regeneration in the aged human.

In yet another aspect, the present invention also comprises pharmaceutical product formulation comprising the compounds disclosed herein. Routes of administration and dosages of effective amounts of the pharmaceutical compositions comprising the compounds are also disclosed. The compounds of the present invention can be administered as liquids such as using an oral syringe or in combination with a capsule or hard or soft gel capsule for effective delivery and treatment of disease in a subject in need thereof, such as older humans.

In one aspect, a pharmaceutical composition comprising an API in a lipid formulation, wherein the API is a compound of formula (I) (as provided below) or a pharmaceutically acceptable salt thereof is disclosed.

In some embodiments, the compound is one of the following compounds or physiologically acceptable salts thereof:

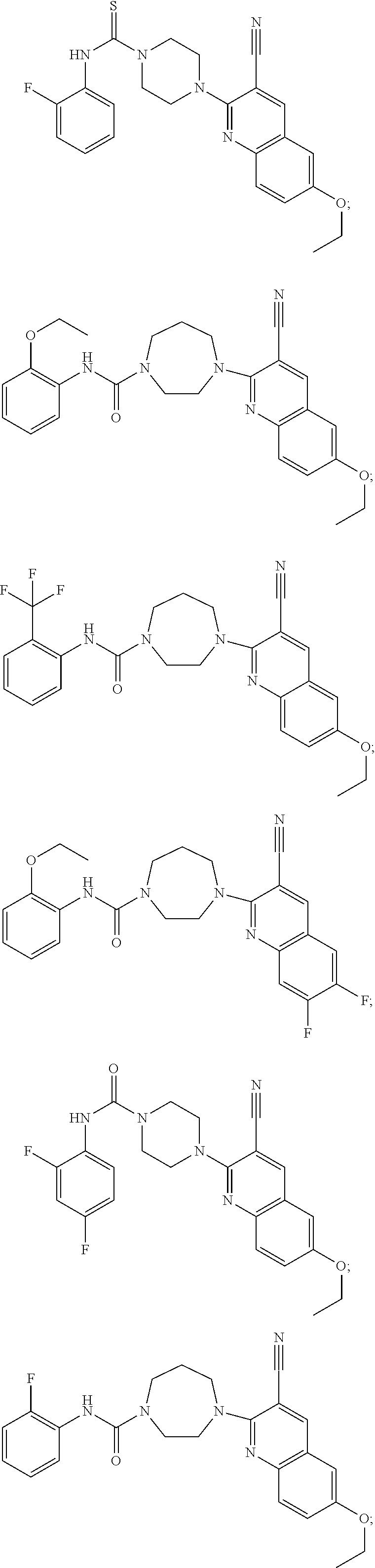

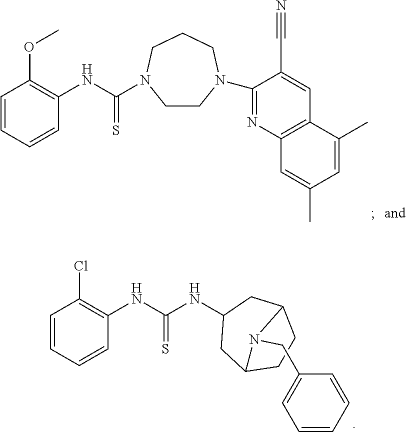 [SMILES: COc1ccccc1NC(=S)N1CCCN(c2nc3cc(C)cc(C)c3cc2C#N)CC1 ; S=C(Nc1ccccc1Cl)NC1CC2CCC(C1)N2Cc1ccccc1]

In some embodiments, the compound is

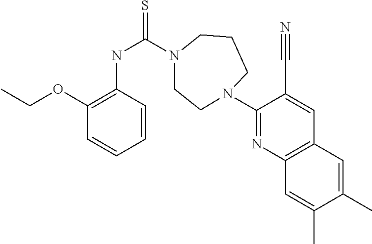 [SMILES: CCOc1ccccc1NC(=S)N1CCCN(c2nc3cc(C)c(C)cc3cc2C#N)CC1]

or a physiologically acceptable salt thereof.

In some embodiments, the compound is

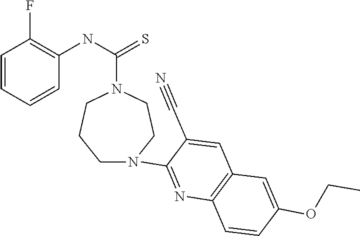

or a physiologically acceptable salt thereof.

In some embodiments, the lipid formulation includes from about 85-95.5% of a vehicle, about 1-8% of a surfactant and about 0.5-7% of a thickening agent by weight of the lipid formulation.

In some embodiments, the vehicle includes one or more of vegetable oils, triglycerides, monoglycerides, diglycerides, oily fatty acids, isopropyl myristate, oily fatty alcohols, esters of sorbitol and fatty acids, oily sucrose esters, mineral oils, and mixtures thereof.

In some embodiment, the vehicle is a medium chain triglyceride (MCT).

In some embodiments, the surfactant is selected from the group consisting of sodium lauryl sulfate, esters of polyoxyethylene sorbitane or polyethylene glycol, sodium dioctylsulfosuccinate (DOSS), lecithin, stearylic alcohol, cetostearylic alcohol, cholesterol, hydrogenated castor oil and derivatives or fractions thereof, Polyoxyl 35 castor oil, Polyoxyl 40 hydrogenated castor oil, hydrogenated polyoxyethylene fatty acid glycerides, pluronic surfactants and mixtures thereof.

In some embodiments, the surfactant is a non-ionic surfactant with an HLB value of 12-14.

In some embodiments, the surfactant is Polyoxyl 35 castor oil.

In some embodiments, the thickening agent is selected from the group consisting of waxes, polyacrylate and polyacrylate co-polymer resins, celluloses and cellulose derivatives and salts thereof, polyvinylpyrrolidones, polyvinyl resins, other polymeric materials, inorganic thickening agents, and mixtures thereof.

In some embodiments, the thickening agent is selected from the group consisting of colloidal silica, bees wax, micro-crystalline wax and mixtures thereof.

In some embodiments, the thickening agent is bees wax.

In some embodiments, the vehicle is a medium chain triglyceride (MCT), the surfactant is Polyoxyl 35 castor oil and the thickening agent is bees wax.

In accordance with another aspect, a method for delivering an API to a mammal is disclosed. The pharmaceutical compositions disclosed herein disposed in a capsule or gel capsule are administered to the mammal.

In accordance with another aspect, a method for treating at least one condition selected from the group consisting of developmental delay, psychiatric disorders, neurodegenerative disease, neurological disorders, and aging in a subject in need thereof is disclosed, wherein the method includes administering to the subject a therapeutically effective amount of a pharmaceutical composition disclosed herein.

In some embodiments, the subject is a mammal. In some embodiments, is a pet or farm animal. In other embodiments, the mammal is a human.

### DETAILED DESCRIPTION

It is understood that the present invention is not limited to the particular methodologies, assays, etc. described herein, as these may vary. It is also to be understood that the terminology used herein is used for the purpose of describing particular embodiments only and is not intended to limit the scope of the present invention.

Unless defined otherwise, all technical and scientific terms used herein have the same meanings as commonly understood by one of ordinary skill in the art to which this invention belongs. Exemplary methods and compositions are described, although any methods and compositions similar or equivalent to those described herein can be used in the practice or testing of the present invention.

### I. Definitions

As used herein, the term “compound” refers to all of the iterations of the structure and formula disclosed herein and also includes a reference to a physiologically acceptable salt thereof. Examples of physiologically acceptable salts of the compounds of the present invention include salts derived from an appropriate base, such as an alkali metal, such as sodium, and alkaline earth, such as magnesium, ammonium and NX₄⁺ (wherein X is C₁-C₄ alkyl). Physiologically acceptable salts of a hydrogen atom or an amino group may include, but are not limited to, salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of a hydroxy group include, but are not limited to, the anion of the compound in combination with a suitable cation such as Na⁺ and NX₄⁺ (wherein X is independently selected from H or a C₁-C₄ alkyl group).

For therapeutic use, salts of the compounds of the present invention will be physiologically acceptable, i.e., the salts will be derived from a physiologically acceptable acid or base. Salts of acids or bases, however, which are not physiologically acceptable may also find use in the preparation or purification of a physiologically acceptable compound. Thus, all salts, whether or not derived form a physiologically acceptable acid or base, are within the scope of the present invention.

“Alkyl” is C₁-C₁₈ hydrocarbon containing normal, secondary, tertiary or cyclic carbon atoms. “Alkenyl” is C₂-C₁₈ hydrocarbon containing normal, secondary, tertiary or cyclic carbon atoms with at least one site of unsaturation, i.e., a carbon-carbon, sp² double bond. Examples include, but are not limited to, ethylene or vinyl (—CH══CH₂), allyl (—CH₂CH══CH₂), cyclopentenyl (— C₅H₇), and 5-hexenyl (— CH₂CH₂CH₂CH₂CH══CH₂). “Alkynyl” is C₂-C₁₈ hydrocarbon containing normal, secondary, tertiary or cyclic carbon atoms with at least one site of unsaturation, i.e., a carbon-carbon, sp triple bond. Examples include, but are not limited to, acetylenic (—C═—CH) and propargyl (—CH₂C≡-CH).

“Aryl” means a monovalent aromatic hydrocarbon radical of 6-20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like.

“Heteroaryl” means a monovalent aromatic radical of one or more carbon atoms and one or more atoms selected from N, O, S, or P, derived by the removal of one hydrogen atom from a single atom of a parent aromatic ring system. Heteroaryl groups may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S). Heteroaryl bicycles have 7 to 10 ring atoms (6 to 9 carbon atoms and 1 to 2 heteroatoms selected from N, O, and S) arranged as a bicyclo [4,5], [5,5], [5,6], or [6,6] system; or 9 to 10 ring atoms (8 to 9 carbon atoms and 1 to 2 hetero atoms selected from N and S) arranged as a bicyclo [5,6] or [6,6] system. The heteroaryl group may be bonded to the drug scaffold through a carbon, nitrogen, sulfur, phosphorus or other atom by a stable covalent bond. Heteroaryl groups include pyridyl, dihydropyridyl isomers, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

Substituted substituents such as “substituted alkyl,” “substituted aryl,” and “substituted heteroaryl,” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, — NR₃, ══NR, —CX₃, —CN, —OCN, —SCN, —N══C══O, —NCS, —NO, —NO₂, ══N₂, —N₃, NC(══O)R, —C(—O)R, —C(══O)NRR—S(══O)₂O⁻, —S(══O)₂OH, —S(══O)₂R, —OS(══O)₂OR, —S(══O₂NR, —S(══O)R, —OP(══O)O₂RR, —P(══O)O₂RR—P(══O)(O⁻)₂, —P(══O)(OH)₂, —C(══O)R, —C(══O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen: F, Cl, Br, or I; and each R is independently —H, alkyl, aryl, heterocycle, protecting group or prodrug moiety. Alkylene, alkenylene, and alkynylene groups may also be similarly substituted.

“Halogens” includes F, Cl, Br or —I and is used interchangeably with the word “halo.”

“Heterocycle” means a saturated, unsaturated or aromatic ring system including at least one N, O, S, or P. Heterocycle thus includes heteroaryl groups. Heterocycle as used herein includes, but is not limited to heterocycles described in PAQUETTE, PRINCIPLES OF MODERN HETEROCYCLIC CHEMISTRY (W. A. Benjamin, New York, 1968), particularly Chapters 1, 3, 4, 6, 7, and 9; THE CHEMISTRY OF HETEROCYCLIC COMPOUNDS, A SERIES OF MONOGRAPHS (John Wiley & Sons, New York, 1950 to present), in particular Volumes 13, 14, 16, 19, and 28; KATRITZKY ET AL., COMPREHENSIVE HETEROCYCLIC CHEMISTRY (Pergamon Press, 1996); and 82 J. AM. CHEM. SOC. 5566 (1960).

Heterocycles include, but are not limited to, pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, .beta.-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

Carbon bonded heterocycles include but are not limited to those that are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

Nitrogen bonded heterocycles include but are not limited to those that are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl. “Carbocycle” means a saturated, unsaturated or aromatic ring system having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo[4,5], [5,5], [5,6] or [6,6] system, or 9 or 10 ring atoms arranged as a bicyclo [5,6] or [6,6] system. Monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl. Carbocycle thus includes aryl groups.

As used herein, the term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow MCGRAW-HILL DICTIONARY OF CHEMICAL TERMS (S. P. Parker, Ed., McGraw-Hill Book Company, New York, 1984); and ELIEL, E. AND WILEN, S., STEREOCHEMISTRY OF ORGANIC COMPOUNDS (John Wiley & Sons, Inc., New York, 1994). Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or I meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The terms “treatment,” “treating,” “treat,” “therapy,” “therapeutic,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The term “pharmaceutically acceptable carrier,” as used herein, refers to any and all solvents, dispersion media, coatings, antibacterial and antifungal agent, isotonic and absorption delaying agents for pharmaceutical active substances as are well known in the art. Except insofar as any conventional media or agent is incompatible with the compound, its use in the therapeutic compositions is contemplated. Supplementary compounds can also be incorporated into the compositions.

As used herein, the term “excipient” refers to the additives used to convert an active compound into a form suitable for its intended purpose. For compositions of the present invention suitable for administration to a human, the term “excipient” includes those excipients described in the HANDBOOK OF PHARMACEUTICAL EXCIPIENTS, American Pharmaceutical Association, 2nd Ed. (1994), which is herein incorporated in its entirety. The term “excipients” is meant to include fillers, binders, disintegrating agents, lubricants, solvents, suspending agents, dyes, extenders, surfactants, auxiliaries and the like. Liquid excipients can be selected from various oils, including those of petroleum, animal, vegetable or synthetic origin, such as, peanut oil, soybean oil, mineral oil, sesame oil, hydrogenated vegetable oil, cottonseed oil, groundnut oils, corn oil, germ oil, olive oil, or castor oil, and so forth.

Suitable excipients also include, but are not limited to, fillers such as saccharides, lactose, fructose, sucrose, inositol, mannitol or sorbitol, xylitol, trehalose, cellulose preparations and/or calcium phosphates, tricalcium phosphate or calcium hydrogen phosphate, as well as starch paste, using modified starch, maize starch, wheat starch, rice starch, potato starch, gelatin, tragacanth, ethoxylated isostearyl alcohols, polyoxyethylene sorbitol and sorbitan esters, microcrystalline cellulose, hydroxypropyl cellulose, methyl cellulose, hydroxypropyl methyl cellulose, aluminum metahydroxide, bentonite, sodium carboxymethylcellulose, croscarmellose sodium, crospovidone and sodium starch glycolate, and/or polyvinyl pyrrolidine and mixtures thereof. If desired, disintegrating agents can be added, such as, the above-mentioned starches and also carboxymethyl-starch, cross-linked polyvinyl pyrrolidone, agar, or alginic acid or a salt thereof, such as, sodium alginate. Auxiliaries include, but are not limited to, silica, stearic acid or salts thereof, such as, magnesium stearate, sodium stearyl fumarate, or calcium stearate.

The expression “therapeutically effective amount” refers to an amount of a compound disclosed herein, that is effective for preventing, ameliorating, treating or delaying the onset of a disease or condition.

The pharmaceutical compositions of the inventions can be administered to any animal that can experience the beneficial effects of the compounds of the invention. Such animals include humans and non-humans such as primates, pets and farm animals.

### II. Active Pharmaceutical Ingredients (API)

In accordance with certain aspects, the pharmaceutical compositions disclosed herein may be used to administer various APIs. Some APIs are disclosed in U.S. Pat. No. 8,999,972, the contents of which are hereby incorporated by reference. Examples of particularly useful APIs include compounds of the formula:

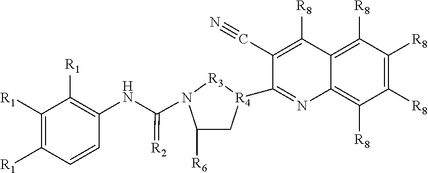

wherein:
each R₁ is independently selected from the group consisting of H, F, Cl, Br, R₇, and —O—R₇, wherein R₇ is a substituted 1-6 carbon alkyl or a 6-14 carbon aryl or aralkyl group;
R₂ is selected from O or S;
R₃ is (CH₂)ₘ, wherein m is 1, 2 or 3;
R₄ is selected from the group consisting of an N and (CHₙ), wherein n equals 1 or 2, with the proviso that when R₄ is nitrogen then m in R₃ should not be equal to 1;
R₆ is H;
each R₈ is independently —X, —R₉, —OR₉, —SR₉, —N(R₉)₂, —CN, —NO₂, —NC(O)R₉, —C(O)R₉, —C(O)N(R₉)₂, —S(O)₂R₉, —S(O)₂NR₉, —S(O)R₉, —C(O)R₉, —C(O)OR₉, or —C(O)N(R₉)₂;
   wherein each X is independently a halogen;
   each R₉ is independently —H, alkyl, alkenyl, alkynyl, aryl, heterocycle, protecting group or prodrug moiety.

More particularly, specific examples of useful compounds include, but are not limited to, the following compounds and physiologically acceptable salts thereof:

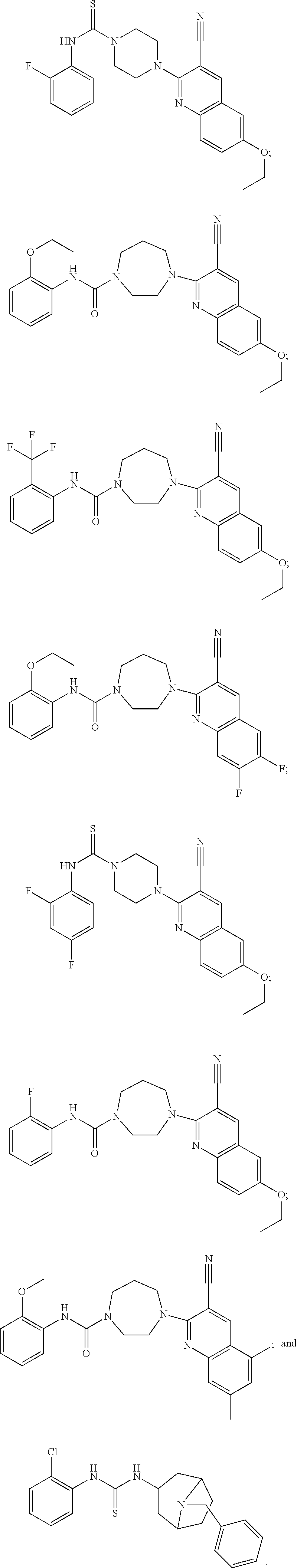

In accordance with one aspect, NNI-362, a compound having the structure:

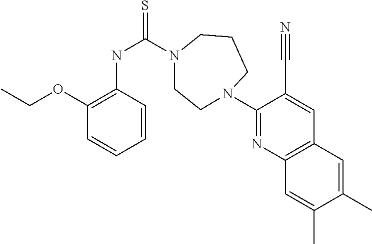 [SMILES: CCOc1ccccc1NC(=S)N1CCCN(c2nc3cc(C)c(C)cc3cc2C#N)CC1]

and physiologically acceptable salts thereof may be used. NNI-362 is a faint yellow to yellow solid that is soluble in common organic solvents, but insoluble in water/aqueous solutions. NNI-362 begins to melt at 152.4° C.

In accordance with another aspect, NNI-351, a compound having the structure:

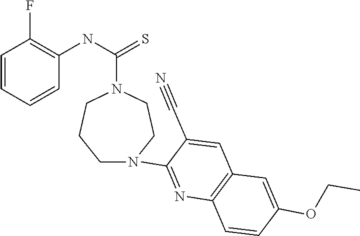

and physiologically acceptable salts thereof may be used.

Finally, the general structure of the compounds of the present invention may encompass all states of saturation of the substituents shown, such as all ene, diene, triene, and yne derivatives of any substituent. The general structures also encompass all conformational isomers, regioisomers, and stereoisomers that may arise from a particular set of substituents. The general structures also encompass all enantiomers, diastereomers, and other optical isomers whether in enantiomeric or racemic forms, or mixtures of stereoisomers.

### III. Pharmaceutical Compositions

The present invention also comprises pharmaceutical compositions comprising the compounds disclosed herein. Routes of administration and dosages of effective amounts of the pharmaceutical compositions comprising the compounds are also disclosed. The compounds of the present invention can be administered in combination with other pharmaceutical agents in a variety of protocols for effective treatment of disease.

The pharmaceutical compositions of the inventions can be administered to any animal that can experience the beneficial effects of the compounds of the invention. Such animals include humans and non-humans such as pets and farm animals (e.g., working animals/livestock/sled dogs).

The pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired.

In addition to the compounds disclosed herein, the pharmaceutical compositions of the present invention may further comprise at least one of any suitable auxiliaries including, but not limited to, diluents, binders, stabilizers, buffers, salts, lipophilic solvents, preservatives, adjuvants or the like. Pharmaceutically acceptable auxiliaries are preferred. Examples and methods of preparing such sterile solutions are well known in the art and can be found in well known texts such as, but not limited to, REMINGTON'S PHARMACEUTICAL SCIENCES (Gennaro, Ed., 18th Edition, Mack Publishing Co. (1990)). Pharmaceutically acceptable carriers can be routinely selected that are suitable for the mode of administration, solubility and/or stability of the compound.

Pharmaceutical excipients and additives useful in the present invention can also include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

Carbohydrate excipients suitable for use in the present invention include monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), myoinositol and the like.

The composition further can contain, but is not limited to, pharmaceutically acceptable carriers or vehicles, such as oil, medium chain triglycerides, coloring agents, emulsifying agents, suspending agents, ethanol, EDTA, citrate buffer, flavoring, and water. More particularly, examples of vehicles include, but are not limited to, a vegetable oil, for example, castor oil, olive oil, soy oil, sesame oil, cotton seed oil, sweet almond oil or arachis oil; triglycerides, such as, for example, semi-synthetic oils (medium chains triglycerides (MCT) or long chain triglycerides); monoglycerides; diglycerides; oily fatty acids; isopropyl myristate; oily fatty alcohols; esters of sorbitol and fatty acids, oily sucrose esters, or a mineral oil, for example, liquid paraffin or petrolatum; and in general any oily substance which is physiologically tolerated and mixtures thereof.

In an embodiment, the vehicle comprises MCT, i.e., a triglyceride oil in which the carbohydrate chain has about 8-12 carbon atoms.

The composition may also include pharmaceutically acceptable thickening agents. Examples of thickening agents include, but are not limited to, waxes (e g., bees wax, carnauba), polyacrylate and polyacrylate co-polymer resins (e.g., Carbopol), celluloses and cellulose derivatives and salts thereof, polyvinylpyrrolidones, polyvinyl resins, other polymeric materials (e.g., gum traganth, gum arabicum, alginates), inorganic thickening agents (e.g., atapulgite, bentonite and silicates). Examples of particularly useful thickening agents include, but are not limited to, colloidal silica, bees wax and micro-crystalline wax.

The composition may further contain one or more surfactants. Examples of useful surfactants include, but are not limited to, sodium lauryl sulfate, esters of polyoxyethylene sorbitane or polyethylene glycol (such as Gelucire), sodium dioctylsulfosuccinate (DOSS), lecithin, stearylic alcohol, cetostearylic alcohol, cholesterol, hydrogenated castor oil and derivatives or fractions thereof, Cremophor or Kolliphor of different grades such as, Cremophore EL/Kolliphor EL (Macrogolglycerol ricinoleate or Polyoxyl 35 castor oil) and Cremophor or Kolliphor RH-40 (Macrogolglycerol hydroxystearate or Polyoxyl 40 hydrogenated castor oil) hydrogenated polyoxyethylene fatty acid glycerides, and pluronic surfactants. Kolliphor® EL is a particularly useful surfactant in the present formulation. Kolliphor EL is a nonionic solubilizer and emulsifier made by reacting castor oil with ethylene oxide. Kolliphor® EL is polyethylene oxide surfactant and is viscous in nature. It has a hydrophobic-lipophilic balance (HLB) value in the range of 12-14.

The composition of the invention also can contain the preservatives methylparaben (also known as 4-hydroxybenzoic acid methyl ester; methyl p-hydroxybenzoate; or METHYL CHEMOSEPT), ethylparaben (also known as 4-hydroxybenzoic acid ethyl ester; ethyl p-hydroxybenzoate; or ETHYL PARASEPT), propylparaben (also known as 4-hydroxybenzoic acid propyl ester; propyl p-hydroxybenzoate; NIPASOL; or PROPYL CHEMOSEPT) and/or butylparaben (also known as 4-hydroxybenzoic acid propyl ester; propyl p-hydroxybenzoate; or BUTYL CHEMOSEPT). In some embodiments, the composition contains methylparaben and/or propylparaben.

Emulsifiers of the invention include, but are not limited to ethyl alcohol, isopropyl alcohol, ethyl carbonate, ethyl acetate, benzyl alcohol, benzyl benzoate, propylene glycol, 1,3-butylene glycol, dimethyl formamide, oils, glycerol, tetrahydrofurfuryl alcohol, polyethylene glycols and fatty acid esters of sorbitan, and mixtures thereof.

The pharmaceutical compositions comprising the compounds disclosed herein can also include a buffer or a pH adjusting agent. Typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

Additionally, the pharmaceutical compositions can include polymeric excipients/additives such as polyvinylpyrrolidones, ficolls (a polymeric sugar), dextrates (e.g., cyclodextrins, such as 2-hydroxypropyl-ß-cyclodextrin), polyethylene glycols, flavoring agents, anti-microbial agents, sweeteners, antioxidants, anti-static agents, surfactants (e.g., polysorbates such as “TWEEN 20” and “TWEEN 80”), lipids (e.g., phospholipids, fatty acids), steroids (e.g., cholesterol), and chelating agents (e.g., EDTA or EGTA). These and additional known pharmaceutical excipients and/or additives suitable for use in the present invention are known in the art, e.g., as listed in REMINGTON: THE SCIENCE & PRACTICE OF PHARMACY (19^{th} ed., Williams & Williams (1995)) and PHYSICIAN'S DESK REFERENCE (52^{nd} ed., Medical Economics (1998)), the disclosures of which are expressly entirely incorporated herein by reference.

The present invention provides stable pharmaceutical compositions as well as preserved solutions and compositions containing a preservative, as well as multi-use preserved compositions suitable for pharmaceutical or veterinary use, comprising at least one compound disclosed herein in a pharmaceutically acceptable composition. Pharmaceutical compositions in accordance with the present invention may optionally contain at least one known preservative. Preservatives include, but are not limited to, phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein. Non-limiting examples include, no preservative, 0.1-2% m-cresol, 0.1-3% benzyl alcohol, 0.001-0.5% thimerosal, 0.001-2.0% pheno, 0.0005-1.0% alkylparaben(s), and the like.

Other excipients, e.g., isotonicity agents, buffers, antioxidants, preservative enhancers, can be optionally added to the diluent. An isotonicity agent such as glycerin, is commonly used at known concentrations. A physiologically tolerated buffer is preferably added to provide improved pH control. The pharmaceutical compositions can cover a wide range of pHs, such as from about pH 4 to about pH 10, specifically, a range from about pH 5 to about pH 9, and more specifically, a range of about 6.0 to about 8.0. In one aspect, the formulations of the present invention have pH between about 6.8 and about 7.8. Suitable buffers include phosphate buffers, sodium phosphate and phosphate buffered saline (PBS).

Other additives, such as a pharmaceutically acceptable solubilizers like Tween 20 (polyoxyethylene (20) sorbitan monolaurate), TWEEN 40 (polyoxyethylene (20) sorbitan monopalmitate), TWEEN 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, PLURONIC® polyls, other block co-polymers, and chelators such as EDTA and EGTA can optionally be added to the pharmaceutical compositions to reduce aggregation. These additives are particularly useful if a pump or plastic container is used to administer the pharmaceutical composition. The presence of pharmaceutically acceptable surfactant mitigates the propensity for the composition to aggregate.

The composition of the invention also can contain the preservatives methylparaben (also known as 4-hydroxybenzoic acid methyl ester; methyl p-hydroxybenzoate; or METHYL CHEMOSEPT), ethylparaben (also known as 4-hydroxybenzoic acid ethyl ester; ethyl p-hydroxybenzoate; or ETHYL PARASEPT), propylparaben (also known as 4-hydroxybenzoic acid propyl ester; propyl p-hydroxybenzoate; NIPASOL; or PROPYL CHEMOSEPT) and/or butylparaben (also known as 4-hydroxybenzoic acid propyl ester; propyl p-hydroxybenzoate; or BUTYL CHEMOSEPT). In some embodiments, the composition contains methylparaben and/or propylparaben.

The compositions of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the present invention, stabilizers, preservatives, excipients, and the like. The preferred lipids are the phospholipids and the phosphatidyl cholines (lecithins), both natural and synthetic. Methods to form liposomes are known in the art (see Prescott, ed., METH. CELL BIOL. 14:33 (1976)). Liposomes, methods of making and methods of use are described in U.S. Pat. No. 4,089,8091 (process for the preparation of liposomes), U.S. Pat. No. 4,233,871 (methods regarding biologically active materials in lipid vesicles), U.S. Pat. No. 4,438,052 (process for producing mixed micelles), U.S. Pat. No. 4,485,054 (large multilamellar vesicles), U.S. Pat. No. 4,532,089 (giant-sized liposomes and methods thereof), U.S. Pat. No. 4,897,269 (liposomal drug delivery system), U.S. Pat. No. 5,820,880 (liposomal formulations), and so forth.

During any of the processes for preparation of the compounds of the present invention, it may be necessary and/or desirable to protect sensitive or reactive groups on any of the molecules concerned. This may be achieved by means of conventional protecting groups, such as those described in PROTECTIVE GROUPS IN ORGANIC CHEMISTRY (1973); and GREENE AND WUTS, PROTECTIVE GROUPS IN ORGANIC SYNTHESIS (1991). The protecting groups may be removed at a convenient subsequent stage using methods known from the art.

The compounds disclosed herein can be solubilized or suspended in a preconcentrate (before dilutions with a diluent), added to the preconcentrate prior to dilution, added to the diluted preconcentrate, or added to a diluent prior to mixing with the preconcentrate. The compounds can also be co-administered as part of an independent dosage form, for therapeutic effect. Optionally, the compounds disclosed herein can be present in a first, solubilized amount, and a second, non-solubilized (suspended) amount.

The pharmaceutical formulation can also contain suitable pharmaceutically acceptable carriers comprising excipients and auxiliaries that facilitate processing of the active compounds into preparations that can be administered to animals, as described herein.

For oral administration in the form of a tablet or capsule, a compound may be combined with an oral, non-toxic pharmaceutically acceptable inert carrier such as ethanol, glycerol, water and the like. Moreover, when desired or necessary, suitable binders, lubricants, disintegrating agents, and coloring agents may also be incorporated into the mixture. Suitable binders include, without limitation, starch; gelatin; natural sugars such as glucose or beta-lactose; corn sweeteners; natural and synthetic gums such as acacia, tragacanth, or sodium alginate, carboxymethylcellulose; polyethylene glycol; waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For oral administration, the composition also optionally contains a sweetener. Sweeteners include but are not limited to sucrose, fructose, sodium saccharin, sucralose (SPLENDA®), sorbitol, mannitol, aspartame, sodium cyclamate, and the like and combinations thereof.

The aqueous suspensions, emulsions and/or elixirs for oral administration of this invention can be combined with various sweetening agents, flavoring agents, such as, but not limited to orange or lemon flavors, coloring agents, such as dye stuffs, natural coloring agents or pigments, in addition to the diluents such as water, glycerin and various combinations, as described herein.

The pharmaceutical compositions of the present invention suitable for oral administration may be presented as discrete units such as capsules, dragees, cachets or tablets each containing a predetermined amount of the compound; as a powder or granules; as a solution or a suspension in an aqueous liquid or a non-aqueous liquid; or as an oil-in-water liquid emulsion or a water-in-oil emulsion, and as a bolus, etc. Liquid formulations can be administered using an oral syringe.

A tablet may be made by compression or molding, optionally with one or more accessory ingredients. Compressed tablets may be prepared by compressing, in a suitable machine, the compound in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the compound therein.

In addition, the compositions comprising compounds may be incorporated into biodegradable polymers allowing for sustained release of the compound. The biodegradable polymers and their uses are described in detail in Brem et al., 74 J. NEUROSURG. 441-46 (1991). Suitable examples of sustained-release compositions include semipermeable matrices of solid hydrophobic polymers containing a compound of the present invention, which matrices are in the form of shaped articles, e.g., films, or microcapsules. Examples of sustained-release matrices include polyesters, hydrogels (including poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT® (Tap Pharmaceuticals, Inc., Chicago, Ill.) (injectable microspheres composed of lactic acid glycolic acid copolymer and leuprolide acetate), and poly-D-(−)-3-hydroxybutyric acid.

Pharmaceutical compositions suitable for parenteral administration include aqueous and non-aqueous sterile injection solutions which may contain anti-oxidants, buffers, bacteriostats and solutes that render the formulation isotonic with the blood of the intended recipient; and aqueous and non-aqueous sterile suspensions which may include suspending agents and thickening agents. The compositions may be presented in unit-dose or multi-dose containers, sealed ampules and vials, and may be stored in a freeze-dried (lyophilized) condition requiring only the addition of the sterile liquid carrier, water for injections, immediately prior to use. Extemporaneous injection solutions and suspensions may be prepared from sterile powders, granules and tablets of the kind previously described.

For parenteral administration, sterile suspensions and solutions are desired. Isotonic preparations which generally contain suitable preservatives are employed when intravenous administration is desired. The pharmaceutical compositions may be administered parenterally via injection of a pharmaceutical composition comprising a compound dissolved in an inert liquid carrier. The term “parenteral,” as used herein, includes, but is not limited to, subcutaneous injections, intravenous, intramuscular, intraperitoneal injections, or infusion techniques. Acceptable liquid carriers include, vegetable oils such as peanut oil, cotton seed oil, sesame oil and the like, as well as organic solvents such as solketal, glycerol formal and the like. The pharmaceutical compositions may be prepared by dissolving or suspending the compound in the liquid carrier such that the final formulation contains from about 0.005% to 30% by weight of a compound.

The composition of the invention can also include additional therapeutic agents such as, but not limited to hydrophilic drugs, hydrophobic drugs, hydrophilic macromolecules, cytokines, peptidomimetics, peptides, proteins, toxoids, sera, antibodies, vaccines, nucleosides, nucleotides, nucleoside analogs, genetic materials and/or combinations thereof.

Examples of therapeutic agents that can be used in the pharmaceutical compositions of the present invention include, but are not limited to, other antineoplastic agents, analgesics and anti-inflammatory agents, anti-anginal agents, antihelmintics, anti arrythmic agents, anti-arthritic agents, anti-asthma agents, anti-bacterial agents, anti-viral agents, antibiotics, anti-coagulants, anti-depressants, anti-diabetic agents, anti-epileptic agents, anti-emetics, anti-fungal agents, anti-gout agents, anti-hypertensive agents, anti-malarial agents, antimigraine agents, anti-muscarinic agents, anti-parkinson's agents, anti-protozoal agents, anti-thyroid agents, thyroid therapeutic agents, anti-tussives, anxiolytic agents, hypnotic agents, neuroleptic agents, ß-blockers, cardiac inotropic agents, corticosteroids, diuretics, gastrointestinal agents, histamine H-receptors antagonists, immunosuppressants, keratolytics, lipid regulating agents, muscle relaxants, nutritional agents, cytokines, peptidomimetics, peptides, proteins, toxoids, sera, sedatives, sex hormones, sex hormone antagonists or agonists, stimulants antibodies, vaccines, nucleosides, nucleoside analogs and genetic materials. Amphiphilic therapeutic agents and nutritional agents can also be included.

The additional therapeutic agent can be solubilized or suspended in a preconcentrate (before dilutions with a diluent), added to the preconcentrate prior to dilution, added to the diluted preconcentrate, or added to a diluent prior to mixing with the preconcentrate. The additional therapeutic agent can also be co-administered as part of an independent dosage form, for therapeutic effect. Optionally, the additional therapeutic agent(s) can be present in a first, solubilized amount, and a second, non-solubilized (suspended) amount. Such additional therapeutic agent(s) can be any agent(s) having therapeutic or other value when administered to an animal, particularly to a mammal, such as drugs, nutrients, and diagnostic agents.

In addition to the compound and compositions of the invention, and additional pharmaceutically active agents, the pharmaceutical formulation can also contain suitable pharmaceutically acceptable carriers comprising excipients and auxiliaries that facilitate processing of the active compounds into preparations that can be administered to animals, as described herein.

Pharmaceutical formulations useful in the present invention can contain a quantity of a compound(s) in an amount effective to treat the condition, disorder or disease of the subject being treated.

The invention is also directed to a kit form useful for administration to patients in need thereof. The kit may have a carrier means being compartmentalized in close confinement to receive two or more container means therein, having a first container means containing a therapeutically effective amount of a pharmaceutical composition of the invention and a carrier, excipient or diluent. Optionally, the kit can have additional container mean(s) comprising a therapeutically effective amount of additional agents.

The kit comprises a container for the separate compositions such as a divided bottle or a divided foil packet, however, the separate compositions can also be contained within a single, undivided container. Typically, the kit contains directions for administration of the separate components. The kit form is particularly advantageous when the separate components are preferably administered at different dosage intervals, or when titration of the individual components of the combination is desired by the prescribing physician. The kits of the invention include testing and screening kits and methods, to enable practitioners to measure levels of the active ingredients in bodily fluids. The kits of the invention also include research-grade reagents and kits available for use and purchase by research entities.

In one aspect, the compositions disclosed herein comprise an API in a base composition. In accordance with some aspects, the base composition comprises a vehicle, a surfactant and a thickening agent. The following provides a representative formulation for a base composition for use herein:

| | Exemplary | Exemplary Range |
|---|---|---|
| Component | Component | (% w/w) w/o API |
| Vehicle | Medium Chain | 85-98.5 |
| | Triglyceride | |
| Surfactant | Kolliphor EL | 1-8 |
| Thickening agent | Bees wax | 0.5-7 |

The vehicle can be present in the base formulation in an amount of from about 85% to 98.5% by weight of the base formulation. In some embodiments, the vehicle is present in the base formulation from about 88% to about 97% by weight, from about 90% to about 95% by weight, from about 92% to about 94% by weight, from about 94% to about 96% by weight.

The surfactant can be present in the base formulation in an amount of from about 1% to 8% by weight of the base formulation. In some embodiments, the surfactant is present in the base formulation from about 2% to about 6% by weight, from about 3% to about 5% by weight, from about 2% to about 4% by weight, from about 2% to about 5% by weight.

The thickening agent can be present in the base formulation in an amount of from about 0.5% to 7% by weight of the base formulation. In some embodiments, the thickening agent is present in the base formulation from about 1% to about 6% by weight, from about 2% to about 5% by weight, from about 2% to about 4% by weight, from about 1% to about 3% by weight.

An API can be added to the base formulation to prepare the finished formulation. In accordance with one aspect, the API can be added in an amount of from about 5% to 40% to the base formulation. In some embodiments, the API is present in the composition from about 8% to about 35% by weight, from about 10% to about 30% by weight, from about 12% to about 25% by weight, from about 15% to about 35% by weight, from about 20% to about 30% by weight, from about 22% to about 28% by weight, from about 5% to about 18% by weight, from about 8% to about 15% by weight.

In accordance with some aspects, the relative ratios of the surfactant (e.g., Kolliphor EL) and thickening agent (e.g., bees wax) may be held relatively constant while the relative ratios of vehicle and API are modified.

An exemplary composition in accordance with one aspect is provided below:

| | Exemplary | Formulation F2 | |
|---|---|---|---|
| Functionality | Component | % w/w | mg/capsule |
| Vehicle | Medium chain | 70 | 336 |
| | triglyceride | | |
| Surfactant | Kolliphor EL | 3 | 14.4 |
| Thickening | Bees wax | 2 | 9.6 |
| agent | | | |
| Active | NNI-362 | 25 | 120 |
| ingredient | | | |
| Total | | 100 | 480 |

An exemplary composition in accordance with another aspect is provided below:

| | Exemplary | Formulation |
|---|---|---|
| Functionality | Component | g |
| Vehicle | Medium chain | 47.5 |
| | triglyceride | |
| Surfactant | Kolliphor EL | 1.5 |
| Thickening agent | Bees wax | 1.0 |
| Active ingredient | NNI-351 | Added to 100 mg/ml |
| | | of lipid base |

### IV. Routes of Administration of Compositions

The invention further relates to the administration of at least one compound disclosed herein by the following routes, including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarenal, intraretinal, intraspinal, intrasynovial, intrathoracic, intrauterine, intravesical, bolus, vaginal, rectal, buccal, sublingual, intranasal, iontophoretic means, or transdermal means.

It can be sometimes desirable to deliver the compounds disclosed herein to the subject over prolonged periods of time, for periods of one week to one year from a single administration. Certain medical devices may be employed to provide a continuous intermittent or on demand dosing of a patient. The devices may be a pump of diffusion apparatus, or other device containing a reservoir of drug and optionally diagnostic or monitoring components to regulate the delivery of the drug. Various slow-release, depot or implant dosage forms can be utilized. A dosage form can contain a pharmaceutically acceptable non-toxic salt of a compound disclosed herein that has a low degree of solubility in body fluids, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g., a zinc tannate salt. Additionally, the compounds of the present invention or a relatively insoluble salt such as those just described, can be formulated in a gel, an aluminum monostearate gel with, e.g., sesame oil, suitable for injection. Salts include, but are not limited to, zinc salts, zinc tannate salts, pamoate salts, and the like. Another type of slow-release depot formulation for injection would contain the compound or salt dispersed or encapsulated in a slow degrading, non-toxic, non-antigenic polymer such as a polylactic acid/polyglycolic acid polymer, including the formulations as described in U.S. Pat. No. 3,773,919. The compounds or relatively insoluble salts thereof such as those described above can also be formulated in cholesterol matrix silastic pellets, particularly for use in animals. Additional slow-release, depot or implant formulations, e.g., gas or liquid liposomes are known in the literature. See, e.g., U.S. Pat. No. 5,770,222; SUSTAINED AND CONTROLLED RELEASE DRUG DELIVERY SYSTEMS (1978).

Other examples include provision of the compounds disclosed herein to be administered by sustained release delivery system containing a biodegradable composition. The biodegradable composition may be composed of a biodegradable, water-coagulable, non-polymeric material and a biocompatible, non-toxic organic solvent that is miscible to dispersible in an aqueous medium. The delivery system may be implanted at an implant site causing the solvent to dissipate, disperse or leach from the composition into surrounding tissue fluid through a resulting microporous matrix.

The term “implant site” is meant to include a site, in or on which the non-polymeric composition is applied. Implantation or implant site can also include the incorporation of the pharmaceutical composition comprising at least one compound of the present invention with a solid device. The pharmaceutical composition can be incorporated into a coating on a stent that is implanted into a subject. Additionally, other solid or biodegradeable materials can be used as a substrate on which the pharmaceutical composition is applied. The coated material, comprising the pharmaceutical composition is then implanted, inserted or is adjacent to the subject or patient. The term “biodegradable” means that the non-polymeric material and/or matrix of the implant will degrade over time by the action of enzymes, by simple or enzymatically catalyzed hydrolytic action and/or by other similar mechanisms in the human body. By “bioerodible,” it is meant that the implant matrix will erode or degrade over time due, at least in part, to contact with substances found in the surrounding tissue fluids, cellular action, and the like. By “bioabsorbable,” it is meant that the non-polymeric matrix will be broken down and absorbed within the human body, by a cell, a tissue, and the like.

Non-polymeric materials that can be used in the composition generally are those that are biocompatible, substantially insoluble in water and body fluids, and biodegradable and/or bioerodible. The non-polymeric material is capable of being at least partially solubilized in a water-soluble organic solvent. The non-polymeric materials are also capable of coagulating or solidifying to form a solid implant matrix. The non-polymeric material is combined with a compatible and suitable organic solvent to form a composition that has the desired consistency ranging from watery to viscous to a spreadable putty or paste.

Suitable organic solvents are those that are biocompatible, pharmaceutically-acceptable, and will at least partially dissolve the non-polymeric material. The organic solvent has a solubility in water ranging from miscible to dispersible. Optionally, a pore-forming agent can be included in the composition to generate additional pores in the implant matrix. The pore-forming agent can be any organic or inorganic, pharmaceutically-acceptable substance that is substantially soluble in water or body fluid, and will dissipate from the coagulating non-polymeric material and/or the solid matrix of the implant into surrounding body fluid at the implant site.

The compounds of the present invention are capable of providing a local or systemic biological, physiological or therapeutic effect in the body of an animal. In formulating some pharmaceutical compositions described herein, the compound is preferably soluble or dispersible in the non-polymeric composition to form a homogeneous mixture, and upon implantation, becomes incorporated into the implant matrix. As the solid matrix degrades over time, the compound is capable of being released from the matrix into the adjacent tissue fluid, and to the pertinent body tissue or organ, either adjacent to or distant from the implant site, preferably at a controlled rate. The release of the compound from the matrix may be varied by the solubility of the compound in an aqueous medium, the distribution of the compound within the matrix, the size, shape, porosity, and solubility and biodegradability of the solid matrix. See e.g., U.S. Pat. No. 5,888,533. The amounts and concentrations of ingredients in the composition administered to the patient will generally be effective to accomplish the task intended.

In other embodiments, the compounds of the present invention may be administered by bioactive agent delivery systems containing microparticles suspended in a polymer matrix. The microparticles may be microcapsules, microspheres or nanospheres currently known in the art. The microparticles should be capable of being entrained intact within a polymer that is or becomes a gel once inside a biological environment. The microparticles can be biodegradable or non-biodegradable. Many microencapsulation techniques used to incorporate a bioactive agent into a microparticle carrier are taught in the art. See e.g., U.S. Pat. Nos. 4,652,441; 5,100,669; 4,438,253; and 5,665,428.

A preferred polymeric matrix will be biodegradable and exhibit water solubility at low temperature and will undergo reversible thermal gelation at physiological mammalian body temperatures. The polymeric matrix is capable of releasing the substance entrained within its matrix over time and in a controlled manner. The polymers are gradually degraded by enzymatic or non-enzymatic hydrolysis in aqueous or physiological environments. See e.g., U.S. Pat. No. 6,287,588.

### V. Methods of Preparation

Methods of preparing various pharmaceutical compositions with a certain amount of active ingredients are known, or will be apparent in light of this disclosure, to those skilled in the art. Methods of preparing said pharmaceutical compositions can incorporate other suitable pharmaceutical excipients and their formulations as described in REMINGTON'S PHARMACEUTICAL SCIENCES, Martin, E. W., ed., Mack Publishing Company, 19th ed. (1995).

Methods of preparing the pharmaceutical preparations of the present invention are manufactured in a manner that is known, including conventional mixing, dissolving, or lyophilizing processes. Thus, liquid pharmaceutical preparations can be obtained by combining the active compounds with solid excipients, optionally grinding the resulting mixture and processing the mixture of granules, after adding suitable auxiliaries, if desired or necessary.

### VI. Methods of Treatment

One of ordinary skill in the art will appreciate that a method of administering pharmaceutically effective amounts of the compositions disclosed herein to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosages can also be administered in a patient-specific manner to provide a predetermined concentration of the agents in the blood, as determined by techniques accepted and routine in the art.

### VII. Dosage Determinations

In general, the compounds disclosed herein may be used alone or in concert with other therapeutic agents at appropriate dosages defined by routine testing in order to obtain optimal efficacy while minimizing any potential toxicity. The dosage regimen utilizing a compound of the present invention may be selected in accordance with a variety of factors including type, species, age, weight, sex, medical condition of the patient; the severity of the condition to be treated; the route of administration; the renal and hepatic function of the patient; and the particular compound employed. A physician or veterinarian of ordinary skill can readily determine and prescribe the effective amount of the drug required to prevent, counter, or arrest the progress of the condition.

Optimal precision in achieving concentrations of drug within the range that yields maximum efficacy with minimal toxicity may require a regimen based on the kinetics of the compound's availability to one or more target sites. Distribution, equilibrium, and elimination of a drug may be considered when determining the optimal concentration for a treatment regimen. The dosages of a compound disclosed herein may be adjusted when combined to achieve desired effects. On the other hand, dosages of these various therapeutic agents may be independently optimized and combined to achieve a synergistic result wherein the pathology is reduced more than it would be if either agent were used alone.

In particular, toxicity and therapeutic efficacy of a compound disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Compounds exhibiting large therapeutic indices are preferred except when cytotoxicity of the compound is the activity or therapeutic outcome that is desired. Although compounds that exhibit toxic side effects may be used, a delivery system can target such compounds to the site of affected tissue in order to minimize potential damage to uninfected cells and, thereby, reduce side effects. Generally, the compounds of the present invention may be administered in a manner that maximizes efficacy and minimizes toxicity.

Data obtained from cell culture assays and animal studies may be used in formulating a range of dosages for use in humans. The dosages of such compounds lie preferably within a range of circulating concentrations that include the ED₅₀ with little or no toxicity. The dosage may vary within this range depending upon the dosage form employed and the route of administration utilized. For any compound used in the methods of the present invention, the therapeutically effective dose may be estimated initially from cell culture assays. A dose may be formulated in animal models to achieve a circulating plasma concentration range that includes the IC₅₀ (the concentration of the test compound that achieves a half-maximal inhibition of symptoms) as determined in cell culture. Such information may be used to accurately determine useful doses in humans. Levels in plasma may be measured, by high performance liquid chromatography.

Moreover, the dosage administration of the pharmaceutical compositions of the present invention may be optimized using a pharmacokinetic/pharmacodynamic modeling system. One or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See U.S. Pat. No. 6,747,002, which is entirely expressly incorporated herein by reference.

Methods are known in the art for determining effective doses for therapeutic and prophylactic purposes for the disclosed pharmaceutical compositions or the disclosed drug combinations, whether or not formulated in the same composition. For therapeutic purposes, the term “jointly effective amount,” as used herein, means that amount of each active compound or pharmaceutical agent, alone or in combination, that elicits the biological or medicinal response in a tissue system, animal or human that is being sought by a researcher, veterinarian, medical doctor or other clinician, which includes alleviation of the symptoms of the disease or disorder being treated. For prophylactic purposes (i.e., inhibiting the onset or progression of a disorder), the term “jointly effective amount” refers to that amount of each active compound or pharmaceutical agent, alone or in combination, that inhibits in a subject the onset or progression of a disorder as being sought by a researcher, veterinarian, medical doctor or other clinician. Thus, the present invention further provides combinations of two or more therapeutic agents wherein, (a) each therapeutic agent is administered in an independently therapeutically or prophylactically effective amount; (b) at least one therapeutic agent in the combination is administered in an amount that is sub-therapeutic or subprophylactic if administered alone, but is therapeutic or prophylactic when administered in combination with the second or additional therapeutic agents according to the invention; or (c) both therapeutic agents are administered in an amount that is subtherapeutic or subprophylactic if administered alone, but are therapeutic or prophylactic when administered together. Combinations of three or more therapeutic agents are analogously possible. Methods of combination therapy include co-administration of a single formulation containing all active agents; essentially contemporaneous administration of more than one formulation; and administration of two or more active agents separately formulated.

More specifically, the pharmaceutical compositions may be administered in a single daily dose, or the total daily dosage may be administered in divided doses of two, three, or four times daily. Doses may be administered for one week, one month, or over the course of several months, 3, 6, 9 or 12 months, or intervals known in the art and determined to be clinically relevant. Doses may be continued throughout the life of the patient, or discontinued when clinical judgment warrants. The daily dosage of the compositions may be varied over a wide range from about 0.0001 to about 1,000 mg per patient, per day. The range may more particularly be from about 0.001 mg/kg to 10 mg/kg of body weight per day, about 0.1-100 mg, about 1.0-50 mg or about 1.0-20 mg per day for adults (at about 60 kg). Additionally, the dosages may be about 0.5-10 mg/kg per day, about 1.0-5.0 mg/kg per day, 5.0-10 mg/kg per day, or equivalent doses as determine by a practitioner, to achieve a serum concentration that is clinically relevant.

In the case of injections, it is usually convenient to give by an intravenous route in an amount of about 0.01-30 mg, about 0.1-20 mg or about 0.1-10 mg per day to adults (at about 60 kg). Intravenous doses may include a bolus or a slow dosing. In the case of other animals, the dose calculated for 60 kg may be administered as well.

As a non-limiting example, treatment of humans or animals can be provided as a one-time or periodic dosage of a compound of the present invention 0.0001 to about 1,000 mg per patient, per day. The range may more particularly be from about 0.001 mg/kg to 10 mg/kg of body weight per day, about 0.1-100 mg, about 1.0-50 mg or about 1.0-20 mg per day for adults (at about 60 kg). Additionally, the dosages may be about 0.5-10 mg/kg per day, about 1.0-5.0 mg/kg per day, 5.0-10 mg/kg per day, or equivalent doses as determine by a practitioner, to achieve a serum concentration that is clinically relevant. In accordance with another aspect, the compounds disclosed herein may be delivered at doses up to about 250 mg/kg orally or by other delivery methods.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

### VIII. Methods of Use of the Compounds of the Invention

In another aspect, the present invention is further directed to methods that have utility in the treatment of any diseases associated with neurodegenerative disease or condition. More specifically, the present invention further provides methods for preventing, slowing progression and/or reversing neurodegenerative disease in a mammal. In a specific embodiment, the method may comprise administering to a mammal a composition comprising a compound described herein. The composition comprising a compound described herein may be administered in an amount effective to prevent, ameliorate, treat or delay the onset of a disease or condition in the mammal.

In a further embodiment, a method for treating a mammal afflicted with a neurodegenerative disease or condition may comprise administering an effective amount of a composition comprising a compound described herein to the mammal. In other embodiments, the neurodegenerative disease or condition may be selected from the group consisting of ischemic stroke, traumatic brain injury, acute disseminated encephalomyelitis, amyotrophic lateral sclerosis (ALS), retinitis pigmentosa, mild cognitive impairment, Alzheimer's disease, Pick's disease, senile dementia, progressive supranuclear palsy, subcortical dementias, Wilson disease, multiple infarct disease, arteriosclerotic dementia, AIDS associated dementia, cerebellar degeneration, spinocerebellar degeneration syndromes, Friedreichs ataxia, ataxia telangiectasia, epilepsy-related brain damage, spinal cord injury, restless legs syndrome, Huntington's disease, Parkinson's disease, striatonigral degeneration, cerebral vasculitis, mitochondrial encephalomyopathies, neuronal ceroid lipofuscinosis, spinal muscular atrophies, lysosomal storage disorders with central nervous system involvement, leukodystrophies, urea cycle defect disorders, hepatic encephalopathies, renal encephalopathies, metabolic encephalopathies, porphyria, bacterial meningitis, viral meningitis, meningoencephalitis, prion diseases, poisonings with neurotoxic compounds, Guillain Barre syndrome, chronic inflammatory neuropathies, polymyositis, dermatomyositis and radiation-induced brain damage. Included in the embodiment is neurodegeneration including peripheral neuropathy due to therapeutic administration of cranial irradiation or chemotherapeutic agents.

In another embodiment, a method for treating a mammal afflicted with a neuropsychiatric disease or condition may comprise administering an effective amount of a composition comprising a compound described herein to the mammal. In other embodiments, the neuropsychiatric disease or condition may be selected from the group consisting of anxiety disorders, childhood disorders, eating disorders, mood disorders, cognitive disorders, personality disorders, psychotic disorders, and substance-related disorders.

More specifically, the types of psychiatric diseases/disorders/conditions that may be treated using the compounds of the present invention include anxiety disorders including, but not limited to, acute stress disorder, panic disorder, agoraphobia, social phobia, obsessive-compulsive disorder, posttraumatic stress disorder, and generalized anxiety disorder; childhood disorders including, but not limited to, attention-deficit hyperactivity disorder, Asperger's disorder, autistic disorder, conduct disorder, oppositional defiant disorder, separation anxiety disorder, and Tourette's disorder; eating disorders including, but not limited to, anorexia nervosa, and bulimia nervosa; mood disorders including, but not limited to, major depressive disorder, bipolar disorder (manic depression), cyclothymic disorder, and dysthymic disorder; cognitive disorders including, but not limited to, delirium, multi-infarct dementia, dementia associated with alcoholism, dementia of the Alzheimer type, and dementia; personality disorders including, but not limited to, paranoid personality disorder, schizoid personality disorder, schizotypal personality disorder, antisocial personality disorder, borderline personality disorder, histrionic personality disorder, narcissistic personality disorder, avoidant personality disorder, dependent personality disorder, and obsessive-compulsive personality disorder; psychotic disorders including, but not limited to, schizophrenia, delusional disorder, brief psychotic disorder, schizophreniform disorder, schizoaffective disorder, and shared psychotic disorder; substance-related disorders including, but not limited to, alcohol dependence, amphetamine dependence, cannabis dependence, cocaine dependence, hallucinogen dependence, inhalant dependence, nicotine dependence, opioid dependence, phencyclidine dependence, and sedative dependence.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

### Example 1

Back-Calculated Concentrations of NNI-362 (Exemplary Formulation F2) in Cohort F (120 mg, Single Dose, Day 1) K₃ EDTA Human Plasma Samples from Neuronascent, Inc. Study Number NNI-001

### Examination of Compounds Effectiveness to be Absorbed by an Aged Human

| SRI | Timepoint | Dilution | Concentration | Analytical |
|---|---|---|---|---|
| Patient ID | (Hours) | Factor | (pg/ml) | Run |
| SRI-F-005 | 0 | 1 | < LLOQ | MF12 |
| SRI-F-005 | 0.5 | 1 | 76.8 | MF12 |
| SRI-F-005 | 1 | 1 | 83.4 | MF12 |
| SRI-F-005 | 2 | 1 | 38.8 | MF12 |
| SRI-F-005 | 4 | 1 | 25.3 | MF12 |
| SRI-F-005 | 6 | 1 | 26.7 | MF12 |
| SRI-F-005 | 8 | 1 | 26.3 | MF12 |
| SRI-F-005 | 12 | 1 | < LLOQ | MF12 |
| SRI-F-005 | 24 | 1 | < LLOQ | MF12 |

Exemplary Formulation F2 was prepared by slowly adding NNI-362 into a jar containing 100 mL of lipid-based MCT/Kolliphor EL/Beeswax (95/3/2% w/w) while mixing and then stirring until all powder is wet. The actual weight of NNI-362 was between 11.88 and 12.12 g to provide the nominal concentration of 120 mg/ml.

### Example 2

A Phase 1A, randomized, placebo-controlled, single and multiple dose, dose-escalation study was conducted to evaluate the safety, tolerability and pharmacokinetics of oral NNI-362 in healthy aged volunteers 50 to 72 years of age. Subjects were fasted prior to dose administration. The plasma concentrations of NNI-362 were determined by a validated bioanalytical method using liquid chromatography-tandem mass spectrometry (LC-MS/MS). The analytical range was 25.0-25000 pg/ml, with 0.0500 ml sample volumes. The stable label d5-NNI-362 was used as the internal standard for this assay.

NNI-362 was formulated in 1% (w/v) methylcellulose in sterile water as an oral suspension that was administered to Cohorts A-E. NNI-362 was administered in a lipid-based formulation (medium-chain triglycerides, Kolliphor EL and beeswax; 95/3/2%; NNI-362 added to provide 120 mg/ml) oral suspension) for Cohorts F and G.

### Pharmacokinetic Data Analysis Methods

PK data analysis was performed on the measurable plasma concentrations of NNI-362. The plasma concentration data were evaluated using Phoenix 64® WinNonlin® software (Version 8.3) to perform noncompartmental analysis. Nominal blood collection times were used in data analysis. The dose administered in dose group was entered into the program as mg/subject.

The following PK parameters were determined for NNI-362 plasma concentrations for each subject: observed maximal plasma concentration (Cₘₐₓ), observed time to reach Cₘₐₓ (Tₘₐₓ), area under the plasma concentration time curve up to the last blood collection time (AUCₗₐₛₜ), and extrapolated to infinity (AUCinf), the percent of AUCinf that was extrapolated (% Extrap AUC_{inf}), terminal elimination half-life (t1/2), and mean residence time to infinity (MRT_{inf}). Terminal t1/2, AUC_{inf}, MRT_{inf} could not be calculated in some subjects due to limited data or poor fit (r<0.8) of the terminal phase of the plasma concentration and time curve. Extrapolation of more than 20% of the AUC may result in unreliable values for the terminal parameters.

### PK Results

The correlation coefficient for the terminal phase was determined using WinNonlin. Significant differences in selected pharmacokinetic parameters between the SAD and MAD dose groups of cohorts F and G were evaluated using the t-test (two tail, paired test; p<0.05).

Cohort A received a single oral dose of 10 mg per subject and no plasma samples from these subjects contained quantifiable concentrations of NNI-362. Cohort B received a dose of 20 mg; two subjects had one plasma sample each that had drug concentrations above the lower limit of quantitation (LLOQ=25 pg/ml). Table 1 shows the number of plasma samples that contained NNI-concentrations greater that 25 pg/ml for Cohorts C, D, and E. Exposure to NNI-362 was low in these samples, and the pharmacokinetic data obtained was limited. The Cₘₐₓ values ranged from 15.1 pg/ml (Subject 5006 MAD, 20 mg) to 69.0 pg/ml (Subject 3007 SAD, 60 mg). There were sufficient data points to calculate AUCₗₐₛₜ for 3 subjects and AUC_{inf} for two subjects. The terminal t1/2 was calculated for two subjects—4.09 hr (Subject 3007) and 13.7 hr (Subject 4008). MRT_{inf} values were 9.2 hr and 5.5 hr for 3007 and 4008, respectively.

Due to the low exposure to NNI-362 observed in Cohorts A-E, a lipid based formulation for oral administration of NNI-362 in accordance with one aspect of the present invention was evaluated. Table 2 shows the pharmacokinetic parameters for Cohorts F and G, SAD and MAD phases. All subjects except those that received the placebo had at least 1 sample with plasma concentrations that were greater than the LLOQ. The doses studied were 120 mg (Cohort F, SAD and MAD) and 240 mg (Cohort G, SAD and MAD). Exposure based on AUC values were higher after multiple doses (MAD) than after a single dose. The Tₘₐₓ values in Cohorts F and G ranged from 0.5 to 8 hr and exhibited similar variability for all four treatment regiments as shown in Table 2 and although the mean Tₘₐₓ values for the MAD treatments were slightly higher than the mean for SAD, the difference was not statistically significant. The mean Cₘₐₓ for Cohort F, SAD was 92.4±60.6 pg/ml (and for Cohort G, SAD was 99.0±63.9 ng/ml). The Cₘₐₓ values after multiple doses were higher than following a single dose—129±67.3 ng/ml (Cohort F, MAD) and 239±230 pg/ml (Cohort G, MAD), suggesting a dose dependent increase in plasma concentrations, although the increase was not statistically significant. The AUC_{inf} values were 590±184 hr·pg/ml (Cohort F, SAD); 1323±681 hr·pg/ml (Cohort G, SAD); 2826±1959 hr·pg/ml (Cohort G, MAD); 3426±3298 hr·pg/ml (Cohort F, MAD). These results indicate that exposure to NNI-362 increases with repeat dose administration. The terminal t_{1/2} values were 6.24±1.84 hr (Cohort F SAD); 12.2±10.2 hr (Cohort F, MAD); 2.58±0.25 hr (Cohort G, SAD); and 11.7±8.94 hr (Cohort G, MAD). The t_{1/2} values indicate that NNI-362 is not completely cleared within 24 hr, resulting in accumulation of NNI-362 and higher AUC values after daily administration compared to a single dose, NNI-362. The mean MRT_{inf} values were lowest for Cohort F SAD (9.99±2.55 hr), and higher in the other three groups: 19.2±14.3 hr (Cohort F MAD), 16.7±17.5 hr (Cohort G SAD), and 17.7±13.0 (Cohort G MAD).

### CONCLUSION

Modification of the formulation for NNI-362 increased the plasma concentrations observed in Cohorts F and G, compared to Cohorts A-E. Cohorts D and F were both administered 120 mg NNI-362, but in the two different formulations. Variability among the subjects was substantial but NNI-362 was quantifiable in plasma samples from all Cohort F subjects that were administered NNI-362 and at more timepoints than the subjects in Cohort D that received NNI-362 in the original formulation. Multiple daily doses and increased dose in the lipid formulation also tended to result in higher exposure to NNI-362 based on Cₘₐₓ and AUC values and in longer terminal t_{1/2} and MRT_{inf} values.

TABLE 1
Pharmacokinetic Parameters of NNI-362: Cohort C, D, and E

| | | | | | | | | | Extrap | |
|---|---|---|---|---|---|---|---|---|---|---|
| | | Dose | | t_{1/2} | Tₘₐₓ | Cₘₐₓ | AUCₗₐₛₜ | AUC_{inf} | AUC_{inf} | |
| ID^{a} | #^{b} | (mg) | Phase | (hr) | (hr) | (pg/ml) | (hr · pg/ml) | (hr · pg/ml) | (%) | MRT_{inf} |
| 3003 | 2 | 60 | SAD | NCc | 6 | 46.8 | NC | NC | NC | (hr) |
| 3007 | 5 | 60 | SAD | 4.09 | 6 | 69.0 | 473 | 622 | 24.1 | 9.2 |
| 3008 | 3 | 60 | SAD | NC | 24 | 33.6 | 623 | NC | NC | NC |
| 4005 | 2 | 120 | SAD | NC | 6 | 37.0 | NC | NC | NC | NC |
| 4007 | 2 | 120 | SAD | NC | 6 | 42.6 | NC | NC | NC | NC |
| 4008 | 7 | 120 | SAD | 13.7 | 2 | 60.1 | 414 | 908 | 54.5 | 5.5 |
| 5006 | 1 | 20 | MAD | NC | 12 | 25.1 | NC | NC | NC | NC |

^{a}Subjects not included on this table from Cohorts A-E did not have quantifiable concentrations of the test article in plasma at any timepoint
^{b}Number of plasma samples with NNI-362 with concentrations greater than the LLOQ (25 pg/ml)
cNC, not calculated due to lack of linearity of terminal phase (r < 0.8) or fewer than 3 data points

TABLE 2
Pharmacokinetic Parameters of NNI-362: Cohorts F and G

| | | | | | | | | | | Extrap | |
|---|---|---|---|---|---|---|---|---|---|---|---|
| | Sbjct | | Dose | | t_{1/2} | Tₘₐₓ | Cₘₐₓ | AUCₗₐₛₜ | AUC_{inf} | AUC_{inf} | MRT_{inf} |
| Cohort | ID | Num ^{a} | (mg) | Phase | (hr) | (hr) | (pg/ml) | (hr · pg/ml) | (hr · pg/ml) | (%) | (hr) |
| F | 6001 | 6 | 120 | SAD | 4.45 | 1 | 83.4 | 289 | 458 | 36.9 | 7.35 |
| F | 6002 | 6 | 120 | SAD | 6.15 | 4 | 70.1 | 561 | 800 | 29.9 | 10.2 |
| F | 6003 | 5 | 120 | SAD | 8.13 | 0.5 | 43.5 | 182 | 511 | 64.5 | 12.4 |
| F | 6004 | 0 | 120 | SAD | NA | NA | NA | NA | NA | NA | NA |
| F | 6005 | 0 | 120 | SAD | NA | NA | NA | NA | NA | NA | NA |
| F | 6006 | 4 | 120 | SAD | NC^{c} | 6 | 101 | 555 | NC | NC | NC |
| F | 6007 | 7 | 120 | SAD | NC | 8 | 208 | 1210 | NC | NC | NC |
| F | 6008 | 1 | 120 | SAD | NC | 2 | 48.4 | NC | NC | NC | NC |
| | | | | Mean | 6.24 | 3.58 | 92.4 | 559 | 590 | 43.8 | 9.99 |
| | | | | SD | 1.84 | 2.97 | 60.6 | 400 | 184 | 18.3 | 2.55 |
| F | 6001 | 7 | 120 | MAD | 2.46 | 6 | 193 | 1150 | 1280 | 9.78 | 6.68 |
| F | 6002 | 9 | 120 | MAD | 12.2 | 2 | 144 | 2680 | 3740 | 28.4 | 19.3 |
| F | 6003 | 9 | 120 | MAD | 29.5 | 2 | 208 | 3800 | 8950 | 57.5 | 42.9 |
| F | 6004 | 0 | 120 | MAD | NA | NA | NA | NA | NA | NA | NA |
| F | 6005 | 0 | 120 | MAD | NA | NA | NA | NA | NA | NA | NA |
| F | 6006 | 7 | 120 | MAD | 5.24 | 2 | 63.8 | 525 | 719 | 26.9 | 9.7 |
| F | 6007 | 9 | 120 | MAD | 11.6 | 8 | 126 | 1770 | 2440 | 27.7 | 17.6 |
| F | 6008 | 3 | 120 | MAD | NC | 6 | 40.4 | 179 | NC | NC | NC |
| | | | | Mean | 12.2 | 4.33 | 129 | 1684 | 3426 | 30.0 | 19.2 |
| | | | | SD | 10.5 | 2.66 | 67.3 | 1369 | 3298 | 17.2 | 14.3 |
| G | 7001 | 1 | 240 | SAD | NC | 2 | 52.3 | NC | NC | NC | NC |
| G | 7002 | 0 | 240 | SAD | NA | NA | NA | NA | NA | NA | NA |
| G | 7003 | 3 | 240 | SAD | NC | 2 | 31.6 | 103 | NC | NC | NC |
| G | 7004 | 4 | 240 | SAD | 2.8 | 6 | 130 | 677 | 790 | 14.4 | 7.76 |
| G | 7005 | 0 | 240 | SAD | NA | NA | NA | NA | NA | NA | NA |
| G | 7006 | 4 | 240 | SAD | 2.6 | 1 | 85.7 | 243 | NC | NC | NC |
| G | 7007 | 7 | 240 | SAD | NC | 2 | 84.6 | 1020 | 2090 | 51.1 | 36.9 |
| G | 7008 | 5 | 240 | SAD | 2.30 | 4 | 210 | 880 | 1090 | 19.3 | 5.56 |
| | | | | Mean | 2.58 | 2.83 | 99.0 | 585 | 1323 | 28.3 | 16.7 |
| | | | | SD | 0.25 | 1.83 | 63.9 | 398 | 681 | 19.9 | 17.5 |
| G | 7001 | 9 | 240 | MAD | 26.2 | 2 | 124 | 1990 | 4520 | 54.8 | 39.5 |
| G | 7002 | 0 | 240 | MAD | NA | NA | NA | NA | NA | NA | NA |
| G | 7003 | 5 | 240 | MAD | 4.29 | 2 | 59.0 | 235 | 426 | 43.9 | 7.10 |
| G | 7004 | 9 | 240 | MAD | 5.10 | 6 | 679 | 4000 | 4230 | 5.10 | 9.51 |
| G | 7005 | 0 | 240 | MAD | NA | NA | NA | NA | NA | NA | NA |
| G | 7006 | 9 | 240 | MAD | NC | 1 | 221 | 2610 | NC | NC | NC |
| G | 7007 | 9 | 240 | MAD | 13.5 | 6 | 267 | 2850 | 3980 | 27.7 | 18.8 |
| G | 7008 | 8 | 240 | MAD | 9.24 | 2 | 82.2 | 569 | 975 | 40.5 | 13.5 |
| | | | | Mean | 11.7 | 3.17 | 239 | 2042 | 2826 | 34.4 | 17.7 |
| | | | | SD | 8.94 | 2.23 | 230 | 1431 | 1959 | 19.0 | 13.0 |

^{a} Number of plasma samples with NNI-362 with concentrations greater than the LLOQ (25 pg/ml)
^{b} NA, not applicable. Subjects received placebo
^{c}NC, not calculated due to lack of linearity of terminal phase (r < 0.8) or fewer than 3 data points

Having now described this invention, it will be understood to those of ordinary skill in the art that the same can be performed within a wide and equivalent range of conditions, formulations and other parameters without affecting the scope of the invention or any embodiment thereof. All patents and publications cited herein are fully incorporated by reference in their entirety.

1. A pharmaceutical composition comprising an API in a lipid formulation, wherein the API comprises a compound of formula (I) or a pharmaceutically acceptable salt thereof: 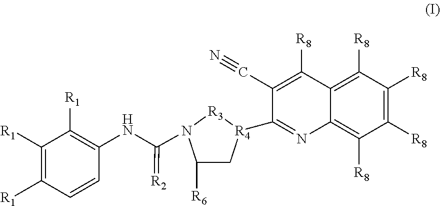
wherein:
each R₁ is independently selected from the group consisting of H, F, Cl, Br, R₇, and —O—R₇, wherein R₇ is a substituted 1-6 carbon alkyl or a 6-14 carbon aryl or aralkyl group;
R₂ is selected from O or S;
R₃ is (CH₂)ₘ, wherein m is 1, 2 or 3;
R₄ is selected from the group consisting of an N and (CHₙ), wherein n equals 1 or 2, with the proviso that when R₄ is nitrogen then m in R₃ should not be equal to 1;
R₆ is H;
each R₈ is independently —X, —R₉, —OR₉, —SR₉, —N(R₉)₂, —CN, —NO₂, —NC(O)R₉, —C(O)R₉, —C(O)N(R₉)₂, —S(O)₂R₉, —S(O)₂NR₉, —S(O)R₉, —C(O)R₉, —C(O)OR₉, or —C(O)N(R₉)₂;
wherein each X is independently a halogen;
each R₉ is independently —H, alkyl, alkenyl, alkynyl, aryl, heterocycle, protecting group or prodrug moiety.

2. The pharmaceutical composition of claim 1, wherein the compound is selected from the group consisting of the following compounds and physiologically acceptable salts thereof:

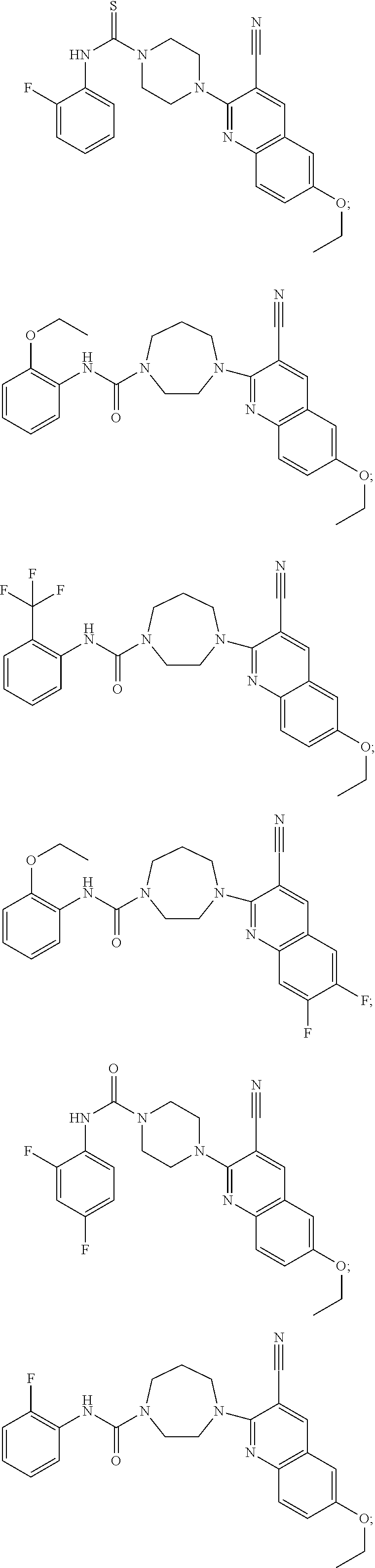

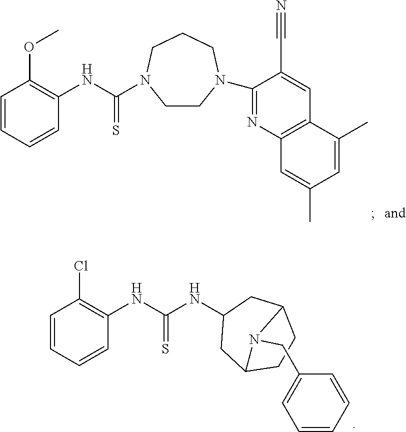 [SMILES: COc1ccccc1NC(=S)N1CCCN(c2nc3cc(C)cc(C)c3cc2C#N)CC1 ; S=C(Nc1ccccc1Cl)NC1CC2CCC(C1)N2Cc1ccccc1]

3. The pharmaceutical composition of claim 1, wherein the compound is 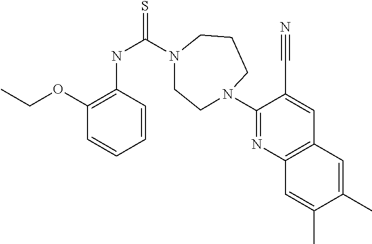 [SMILES: CCOc1ccccc1NC(=S)N1CCCN(c2nc3cc(C)c(C)cc3cc2C#N)CC1]
or a physiologically acceptable salt thereof.

4. The pharmaceutical composition of claim 1, wherein the compound is 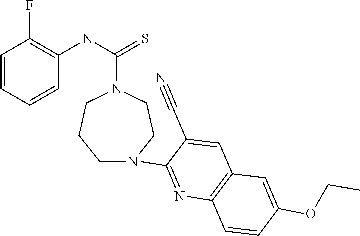
or a physiologically acceptable salt thereof.

5. The pharmaceutical composition of claim 1, wherein the lipid formulation comprises from about 85-95.5% of a vehicle, about 1-8% of a surfactant and about 0.5-7% of a thickening agent by weight of the lipid formulation.

6. The pharmaceutical composition of claim 5, wherein the vehicle is selected from the group consisting of vegetable oils, triglycerides, monoglycerides, diglycerides, oily fatty acids, isopropyl myristate, oily fatty alcohols, esters of sorbitol and fatty acids, oily sucrose esters, mineral oils, and mixtures thereof.

7. The pharmaceutical composition of claim 6, wherein the vehicle is a medium chain triglyceride (MCT).

8. The pharmaceutical composition of claim 5, wherein the surfactant is selected from the group consisting of sodium lauryl sulfate, esters of polyoxyethylene sorbitane or polyethylene glycol, sodium dioctylsulfosuccinate (DOSS), lecithin, stearylic alcohol, cetostearylic alcohol, cholesterol, hydrogenated castor oil and derivatives or fractions thereof, Polyoxyl 35 castor oil, Polyoxyl 40 hydrogenated castor oil, hydrogenated polyoxyethylene fatty acid glycerides, pluronic surfactants and mixtures thereof.

9. The pharmaceutical composition of claim 8, wherein the surfactant is a non-ionic surfactant with an HLB value of 12-14.

10. The pharmaceutical composition of claim 8, wherein the surfactant is Polyoxyl 35 castor oil.

11. The pharmaceutical composition of claim 5, wherein the thickening agent is selected from the group consisting of waxes, polyacrylate and polyacrylate co-polymer resins, celluloses and cellulose derivatives and salts thereof, polyvinylpyrrolidones, polyvinyl resins, other polymeric materials, inorganic thickening agents, and mixtures thereof.

12. The pharmaceutical composition of claim 11, wherein the thickening agent is selected from the group consisting of colloidal silica, bees wax, micro-crystalline wax and mixtures thereof.

13. The pharmaceutical composition of claim 12, wherein the thickening agent is bees wax.

14. The pharmaceutical composition of claim 5, wherein the vehicle is a medium chain triglyceride (MCT), the surfactant is Polyoxyl 35 castor oil and the thickening agent is bees wax.

15. A method for delivering the API to a mammal, comprising administering the pharmaceutical composition of claim 1 in a capsule or gel capsule to the mammal.

16. A method for treating at least one condition selected from the group consisting of developmental delay, psychiatric disorders, neurodegenerative disease, neurological disorders, and aging in a subject in need thereof, the method comprising administering to the subject a therapeutically effective amount of a pharmaceutical composition of claim 1.

17. The method of claim 16, wherein the subject is a mammal.

18. The method of claim 17, wherein the mammal is a pet or farm animal.

19. The method of claim 17 wherein the mammal is a human.
